(19) United States
(12) Patent Application Publication
(10) Pub. No.: US 2006/0165812 A1
(43) Pub. Date: Jul. 27, 2006

(54) Method and topical formulation for treating headaches

(75) Inventor: Dan Charron, Brossard (CA)

Correspondence Address: BCF, LLP, 1100 West Rene'-Levesques Blvd., 25th Floor, Montreal, H3B 5C9, CA

(73) Assignee: Amershire Investment Corporation

(21) Appl. No.: 11/038,101

(22) Filed: Jan. 21, 2005

(51) Int. Cl. A61K 33/00; A61K 33/26; A61K 36/06; A61K 33/42; A61K 33/38; A61K 33/36; A61K 36/282; A61K 36/886; A61K 36/899; A61K 36/28; A61K 36/33

(52) U.S. Cl. 424/600; 424/620; 424/195.16; 424/725; 424/740; 424/744; 424/618; 424/767; 424/776; 424/750; 424/761; 424/764; 424/765; 424/646; 424/603; 424/725.1

(57) ABSTRACT

Disclosed herein are methods, topical formulations and kits for treating, ameliorating or at least reducing pain intensity associated with a headache in a host suffering therefrom. These methods, topical formulations and kits use therapeutically effective amounts of at least one from a plurality of active agents that are directly applied to the head or cephalic region of a host. The active agent comprise a homeopathic ingredient or ingredients known for treating, ameliorating or at least reducing pain intensity associated with a headache in a host suffering therefrom when administered internally.

### FIELD OF THE INVENTION

The present invention relates to the treatment of headaches. More specifically, the present invention is concerned with the topical application of homeopathic active ingredients for treating a patient suffering from a headache.

### BACKGROUND OF THE INVENTION

Headaches are a common problem affecting a large segment of the population. Headaches, such as tension type and migraine headaches, occur both acutely and chronically, and can arise in response to variety of stimulants, including stress, injury, toxins in the environment and the like.

A variety of topical methods, formulations and kits for treating headaches are known in the art. Most of these prior art methods use local anesthetics or allopathic drugs, which may cause secondary effects.

Homeopathic medicine has gained popularity due to its safety, namely that the usage of homeopathic medicines does not bring about the many side effects often associated with allopathic medicines.

There thus remains a need for providing improved methods and formulations for topically treating headaches.

### OBJECTS OF THE INVENTION

An object of the present invention is to provide an improved method and topical formulation for treating headaches.

### SUMMARY OF THE INVENTION

In accordance with the present invention, there is provided a method for treating, ameliorating or at least reducing pain intensity associated with a headache in a host suffering therefrom, the method comprising topically applying a therapeutically effective amount of a homeopathic active agent to the cephalic region of said host, wherein said active agent comprises a homeopathic ingredient known for treating, ameliorating or at least reducing pain intensity associated with a headache in a host suffering therefrom when administered internally.

The active agent being selected from the group consisting of: Absinthium, Aceticum Acidum, Actaea Spicata, Agaricus Muscarius, Aceticum Acid, Aconitum Cammarum, Aconitum Napellus, Aesculus Hippocastanum, Aethusa, Ailanthus Glandulosa, Alfalfa, Allium Cepa, Allium Sativum, Aloe Socotrina, Alumen, Alumina, Ambra Grisea, Ammoniacum Gummi, Ammonium Iodatum, Ammonium Picricum, Amphisbaena, Amygdalae Amarae Aqua, Amylenum Nitrosum, Anacardium Orientale, Anagallis Arvensis, Angustura Vera, Anhalonium Lewinii, Anthemis Nobilis, Antimonium Crudum, Antimonium Tartaricum, Antipyrinum, Aphis Chenopodii Glauci, Apis Mellifica, Apium Graveolens, Apocynum Cannabinum, Apomorphinum, Aqua Marina, Aranea Diadema, Aranea Scinencia, Argemone Mexicana, Argentum Metallicum. Argentum Nitricum, Armoracea Sativa, Arnica, Arsenicum Album, Arsenicum Metallicum, Arsenicum Sulphuratum Flavum, Arum Dracontium, Arum Italicum, Arum Triphyllum, Arundo Mauritanica, Asa Foetida, Asarum Europaeum, Asclepias Cornuti, Asclepias Syriaca, Asclepias Tuberosa, Asparagus Officinalis, Asterias Rubens, Astragalus Menziesii, Astragalus Mollissimus, Athamanta, Aurantium, Aurum Arsenicicum, Aurum Bromatum, Aurum Metallicum, Aurum Muriaticum Natronatum, Avena Sativa, Azadirachta Indica, Bacillinum Burnett, Badiaga, Baptisia Confusa Acetica, Baptisia Tinctoria, ryta Carbonica, Baryta Muriatica, Belladonna, Bellis Perennis, Benzinum, Benzoicum Acidum, Benzolum, Berberis Aquifolium, Berberis Vulgaris, Bismuthum Subnitricum, Boletus Laricis, Boracicum Acidum, Borax Veneta, Bovista Lycoperdon, Bromium, Brucea Antidysenterica, Brucinum, Bryonia Alba, Bufo Rana, Butyricum Acidum, Cactus Grandiflorus, Cadmium Sulphuratum, Cajuputum, Caladium Seguinum, Calcarea Acetica, Calcarea Arsenicosa, Calcarea Carbonica, Calcarea Fluorica, Calcarea Hypophosphorosa, Calcarea Iodata, Calcarea Lactica, Calcarea Phosphorica, Calendula Officinalis, Camphora, Cannabis Indica, Cantharis Vesicatoria, Capsicum Annuum, Carbo Animalis, Carbo Vegetabilis, Carbolicum Acidum, Carboneum Oxygenisatum, Carboneum Sulphuratum, Carduus Benedictus, Carduus Marianus, Carlsbad Aqua, Cascara Sagrada, Causticum, Cedron, Cenchris Contortrix, Cereus Bonplandii, Chamomilla, Chelidonium Majus, Chenopodium Anthelminticum, Chimaphila Maculata, Chimaphila Umbellata, China Officinalis, Chininum Arsenicosum, Chininum Muriaticum, Chininum Sulphuricum, Chionanthus Virginica, Chloralum Hydratum, Cichorium, Cicuta Virosa, Cimex Lectularius, Cimicifuga Racemosa, Cina Maritima, Cinchona Off, Cinchoninum Sulphuricum, Cinnamomum, Clematis Erecta, Clematis Erecta, Cobaltum Metallicum, Coca-Erythroxylon Coca, Cocainum Hydrochloricum, Coccinella Septempunctata, Cocculus Indicus, Coccus Cacti, Cochlearia Armoracia, Codeinum, Coffea Cruda, Colchicum Autumnale, Collinsonia Canadensis, Colocynthinum, Colocynthis, Condurango, Conium Maculatum, Convallaria Majalis, Convolvulus Duartinus, Copaiva Officinalis, Corallium Rubrum, Cornus Circinata, Crataegus Oxyacantha, Crocus Sativus, Crotalus Cascavella, Crotalus Horridus, Croton Tiglium, Cundurango, Cupressus Australis, Cuprum Aceticum, Cuprum Metallicum, Curare, Cyclamen Europaeum, Cypripedium Pubescens, Daphne Indica, Digitalis Purpurea, Dioscorea Villosa, Dirca Palustris, Drosera Rotundifolia, Dulcamara, Echinacea Angustifolia, Elaeis Guineensis, Elaps Corallinus, Electricitas, Ephedra Vulgaris, Epiphegus Virginiana, Eserinum, Eucalyptus Globulus, Eugenia Jambos, Euonymus Atropurpurea, Euonymus Europaea, Eupatorium Perfoliatum, Eupatorium Purpureum, Euphorbia Hypericifolia, Euphorbium Officinarum, Euphrasia Officinalis, Eupionum, Fagopyrum Esculentum, Fagus, Fel Tauri, Ferrum Bromatum, Ferrum Metallicum, Ferrum Muriaticum, Ferrum Phosphoricum, Ferrum Sulphuricum, Ferrum Tartaricum, Feverfew, Ficus Religiosa, Fluoricum Acidum, Formica Rufa, Franciscea Uniflora, Fraxinus Americana, Gallicum Acidum, Gaultheria Procumbens, Gelesemium Sempervirens, Gelsemium Sempervirens, Genista, Gentiana Lutea, Geranium Maculatum, Ginger, Ginseng, Ginseng Quinquefolium, Glonoine, Glonoinum, Glycerinum, Gnaphalium Polycephalum, Goldenseal, Gossypium Herbaceum, Graphites, Gratiola, Gratiola Officinalis, Grindelia Robusta, Guaco, Guaiacum, Guajacum Officinale, Guarana, Guarea Trichiloides, Gymnocladus Canadensis, Haematoxylon, Haematoxylon Campechianum, Hamamelis Virginiana, Hedeoma Pulegioides, Helleborus Niger, Heloderma, Helonias Dioica, Hepar Sulphur, Heracleum Sphondylium, Herbs Dandelion, Hippuricum Acidum, Homarus, Hydrastis Canadensis, Hydrocyanicum Acidum, Hydrophobinum, Hyoscyamus Niger, Hypericum, Hypericum Perforatum, Iberis Amara, Ichthyolum, Ictodes Foetida, Ignatia Amara, Indigo Tinctoria, Indium Metallicum, Indolum, Inula Helenium, lodium, lodoformum, Ipecacuanha, Iridium Metallicum, Iris Florentina, Iris Foetidissima, Iris Tenax, Iris Versicolor, Jacaranda Caroba, Joanesia Asoca, Juglans Cinerea, Juglans Regia, Juncus, Justicia Adhatoda, Kali Carbonicum, Kali Cyanatum, Kali Nitricum, Kali Oxalicum, Kali Phosphoricum, Kalium Bromatum, Kalium lodatum, Kalium Muriaticum, Kalium Silicicum, Kalium Sulphuricum, Kalmia Latifolia, Kreosotum, Lac Caninum, Lac Felinum, Lac Vaccinum Defloratum, Lachesis, Lachnanthes, Lactuca Virosa, Lamium, Lapathum, Latrodectus Mactans, Lecithinum, Ledum Palustre, Leptandra, Lilium Tigrinum, Limulus Cyclops, Lithium Carbonicum, Lobelia Cardinalis, Lobelia Dortmanna, Lobelia Inflata, Lobelia Purpurascens, Loleum Temulentum, Lonicera Xylosteum, Luna, Lupulus Humulus, Lycopersicum, Lycopodium Clavatum, Lycopus, Lyssinum, Macrotinum, Magnesia Muriatica, Magnesia Phosphorica, Magnesium Sulphuricum, Mancinella, Manganum, Medorrhinum, Melilotus Officinalis, Menispermum, Mentha Piperita, Mentha Pulegium, Mentholum, Menyanthes, Mercurialis Perennis, Mercurius Corrosivus, Mercurius Cyanatus, Mercurius Solubilis, Mezereum, Millefolium, Momordica Balsamica, Morphinum, Moschus, Muriaticum Acidum, Myrica Cerifera, Naja Tripudians, Natrium Arsenicosum, Natrium Nitricum, Natrium Phosphoricum, Natrium Salicylicum, Natrum Carbonicum, Natrum Hypochlorosum, Natrum Muriaticum, Natrum Sulphuricum, Niccolum Metallicum, Niccolum Sulphuricum, Nitricum Acidum, Nuphar Luteum, Nux Moschata, Nux Vomica, Nyctanthes Arbor Tristis, Oenanthe Crocata, Oleander, Oleum Animale Aethereum, Oleum Jecoris Aselli, Oniscus Asellus, Ononis, Onosmodium Virginianum, Opium, Oreodaphne, Osmium, Ostrya, Oxalicum Acidum, Oxytropis Lamberti, Paeonia, Palladium, Paraffinum, Paris, Paris Quadrifolia, Parthenium, Passiflora Incarnata, Penthorum Sedoides, Petroleum, Phaseolus, Phellandrium, Phenacetinum, Phosphoricum Acidum, Phosphorus, Physalis Alkekengi, Physostigma, Physostigma Venenosum, Phytolacca, Picricum Acidum, Pimpinella, Piper Methysticum, Piper Nigrum, Plantago Major, Platinum Metallicum, Plumbum Metallicum, Podophyllum, Polyporus, Populus Candicans, Pothos Foetida, Primula Veris, Prunus Padus, Prunus Spinosa, Psorinum, Ptelea,. Pulex Irritans, Pulsatilla Pratensis, Pulsatilla, Pyrogenium, Radium Bromatum, Ranunculus Bulbosus, Ranunculus Glacialis, Ranunculus Sceleratus, Raphanus, Ratanhia Peruviana, Rhamnus Californica, Rheum, Rhodium Oxydatum Nitricum, Rhododendron Ferrugineum, Rhus Glabra, Rhus Toxicodendron, Rhus Venenata, Ricinus Communis, Robinia, Ruta Graveolens, Sabadilla, Sabal Serrulata, Sabina, Saccharum Lactis, Saccharum Officinale, Salicylicum Acidum, Salix Alba, Salol, Sambucus Nigra, Sanguinaria Canadensis, Sanguinarinum Nitricum, Sanicula, Santoninum, Saponaria Officinalis, Saponinum, Sarsaparilla, Scrophularia Nodosa, Scutellaria Laterifolia, Secale Cornutum, Selenium, Senecio Aureus, Senecio Jacoboea, Senega, Sepia Officinalis, Silica, Silicea Terra, Sinapis Alba, Sinapis Nigra, Skatolum, Sol, Solanum Nigrum, Solanum Tuberosum Egrotans, Spigelia Anthelmia, Spigelia Marilandica, Spongia Tosta, Stachys Betonica, Stannum, Staphysagria, Stellaria Media, Sticta Pulmonaria, Stillingia Sylvatica, Stramonium, Strontium Carbonicum, Strontium Nitricum, Strophanthus Hispidus, Strychninum, Succinum, Sulfonal, Sulphur, Sulphuricum Acidum, Sulphurosum Acidum, Sumbulus Moschatus, Symphytum Officinale, Syphilinum, Tabacum, Tanacetum Vulgare, Taraxacum Officinale, Tarentula Cubensis, Tarentula Hispanica, Taxus Baccata, Tellurium Metallicum, Terebinthiniae Oleum, Teucrium Marum Verum, Thea Chinensis, Theridion, Thlaspi Bursa Pastoris, Thuja Occidentalis, Thyroidinum, Tilia Europaea, Tongo, Torula Cerevisiae, Trifolium Pratense, Trillium Pendulum, Trinitrotoluenum, Triosteum Perfoliatum, Tuberculinum, Turnera Aphrodisiaca, Tussilago Petasites, Upas, Upas Tieut, Uranium Nitricum, Urtica Urens, Usnea Barbata, Ustilago, Ustilago Maydis, Vaccininum, Valeriana, Variolinum, Veratrum Album, Veratrum Nigrum, Veratrum Viride, Verbascum Thapsus, Viburnum Opulus, Vinca Minor, Viola Odorata, Viola Tricolor, Viscum Album, Wyethia, Xanthoxylum, Xerophyllum, X-Ray, Yohimbinum, Yucca Filamentosa, Zincum, Zincum Metallicum, Zincum Phosphoricum, Zincum Picricum, Zincum Sulphuricum, Zincum Valerianicum, Zingiber Officinale, Zizia, and any combination thereof.

In accordance with another aspect of the present invention, there is provided a topical formulation for treating, ameliorating or at least reducing pain intensity associated with a headache or a migraine in a host suffering therefrom, the formulation being applicable to the cephalic region of the host, the formulation comprising:

a therapeutically effective amount of an active agent being selected from the group consisting of: Absinthium, Aceticum Acidum, Actaea Spicata, Agaricus Muscarius, Aceticum Acid, Aconitum Cammarum, Aconitum Napellus, Aesculus Hippocastanum, Aethusa, Ailanthus Glandulosa, Alfalfa, Allium Cepa, Allium Sativum, Aloe Socotrina, Alumen, Alumina, Ambra Grisea, Ammoniacum Gummi, Ammonium lodatum, Ammonium Picricum, Amphisbaena, Amygdalae Amaree Aqua, Amylenum Nitrosum, Anacardium Orientale, Anagallis Arvensis, Angustura Vera, Anhalonium Lewinii, Anthemis Nobilis, Antimonium Crudum, Antimonium Tartaricum, Antipyrinum, Aphis Chenopodii Glauci, Apis Mellifica, Apium Graveolens, Apocynum Cannabinum, Apomorphinum, Aqua Marina, Aranea Diadema, Aranea Scinencia, Argemone Mexicana, Argentum Metallicum. Argentum Nitricum, Armoracea Sativa, Arnica, Arsenicum Album, Arsenicum Metallicum, Arsenicum Sulphuratum Flavum, Arum Dracontium, Arum Italicum, Arum Triphyllum, Arundo Mauritanica, Asa Foetida, Asarum Europaeum, Asclepias Cornuti, Asclepias Syriaca, Asclepias Tuberosa, Asparagus Officinalis, Asterias Rubens, Astragalus Menziesii, Astragalus Mollissimus, Athamanta, Aurantium, Aurum Arsenicicum, Aurum Bromatum, Aurum Metallicum, Aurum Muriaticum Natronatum, Avena Sativa, Azadirachta Indica, Bacillinum Burnett, Badiaga, Baptisia Confusa Acetica, Baptisia Tinctoria, ryta Carbonica, Baryta Muriatica, Belladonna, Bellis Perennis, Benzinum, Benzoicum Acidum, Benzolum, Berberis Aquifolium, Berberis Vulgaris, Bismuthum Subnitricum, Boletus Laricis, Boracicum Acidum, Borax Veneta, Bovista Lycoperdon, Bromium, Brucea Antidysenterica, Brucinum, Bryonia Alba, Bufo Rana, Butyricum Acidum, Cactus Grandiflorus, Cadmium Sulphuratum, Cajuputum, Caladium Seguinum, Calcarea Acetica, Calcarea Arsenicosa, Calcarea Carbonica, Calcarea Fluorica, Calcarea Hypophosphorosa, Calcarea lodata, Calcarea Lactica, Calcarea Phosphorica, Calendula Officinalis, Camphora, Cannabis Indica, Cantharis Vesicatoria, Capsicum Annuum, Carbo Animalis, Carbo Vegetabilis, Carbolicum Acidum, Carboneum Oxygenisatum, Carboneum Sulphuratum, Carduus Benedictus, Carduus Marianus, Carlsbad Aqua, Cascara Sagrada, Causticum, Cedron, Cenchris Contortrix, Cereus Bonplandii, Chamomilla, Chelidonium Majus, Chenopodium Anthelminticum, Chimaphila Maculata, Chimaphila Umbellata, China Officinalis, Chininum Arsenicosum, Chininum Muriaticum, Chininum Sulphuricum, Chionanthus Virginica, Chloralum Hydratum, Cichorium, Cicuta Virosa, Cimex Lectularius, Cimicifuga Racemosa, Cina Maritima, Cinchona Off, Cinchoninum Sulphuricum, Cinnamomum, Clematis Erecta, Clematis Erecta, Cobaltum Metallicum, Coca-Erythroxylon Coca, Cocainum Hydrochloricum, Coccinella Septempunctata, Cocculus Indicus, Coccus Cacti, Cochlearia Armoracia, Codeinum, Coffea Cruda, Colchicum Autumnale, Collinsonia Canadensis, Colocynthinum, Colocynthis, Condurango, Conium Maculatum, Convallaria Majalis, Convolvulus Duartinus, Copaiva Officinalis, Corallium Rubrum, Cornus Circinata, Crataegus Oxyacantha, Crocus Sativus, Crotalus Cascavella, Crotalus Horridus, Croton Tiglium, Cundurango, Cupressus Australis, Cuprum Aceticum, Cuprum Metallicum, Curare, Cyclamen Europaeum, Cypripedium Pubescens, Daphne Indica, Digitalis Purpurea, Dioscorea Villosa, Dirca Palustris, Drosera Rotundifolia, Dulcamara, Echinacea Angustifolia, Elaeis Guineensis, Elaps Corallinus, Electricitas, Ephedra Vulgaris, Epiphegus Virginiana, Eserinum, Eucalyptus Globulus, Eugenia Jambos, Euonymus Atropurpurea, Euonymus Europaea, Eupatorium Perfoliatum, Eupatorium Purpureum, Euphorbia Hypericifolia, Euphorbium Officinarum, Euphrasia Officinalis, Eupionum, Fagopyrum Esculentum, Fagus, Fel Tauri, Ferrum Bromatum, Ferrum Metallicum, Ferrum Muriaticum, Ferrum Phosphoricum, Ferrum Sulphuricum, Ferrum Tartaricum, Feverfew, Ficus Religiosa, Fluoricum Acidum, Formica Rufa, Franciscea Uniflora, Fraxinus Americana, Gallicum Acidum, Gaultheria Procumbens, Gelesemium Sempervirens, Gelsemium Sempervirens, Genista, Gentiana Lutea, Geranium Maculatum, Ginger, Ginseng, Ginseng Quinquefolium, Glonoine, Glonoinum, Glycerinum, Gnaphalium Polycephalum, Goldenseal, Gossypium Herbaceum, Graphites, Gratiola, Gratiola Officinalis, Grindelia Robusta, Guaco, Guaiacum, Guajacum Officinale, Guarana, Guarea Trichiloides, Gymnocladus Canadensis, Haematoxylon, Haematoxylon Campechianum, Hamamelis Virginiana, Hedeoma Pulegioides, Helleborus Niger, Heloderma, Helonias Dioica, Hepar Sulphur, Heracleum Sphondylium, Herbs Dandelion, Hippuricum Acidum, Homarus, Hydrastis Canadensis, Hydrocyanicum Acidum, Hydrophobinum, Hyoscyamus Niger, Hypericum, Hypericum Perforatum, Iberis Amara, Ichthyolum, Ictodes Foetida, Ignatia Amara, Indigo Tinctoria, Indium Metallicum, Indolum, Inula Helenium, Iodium, Iodoformum, Ipecacuanha, Iridium Metallicum, Iris Florentina, Iris Foetidissima, Iris Tenax, Iris Versicolor, Jacaranda Caroba, Joanesia Asoca, Juglans Cinerea, Juglans Regia, Juncus, Justicia Adhatoda, Kali Carbonicum, Kali Cyanatum, Kali Nitricum, Kali Oxalicum, Kali Phosphoricum, Kalium Bromatum, Kalium Iodatum, Kalium Muriaticum, Kalium Silicicum, Kalium Sulphuricum, Kalmia Latifolia, Kreosotum, Lac Caninum, Lac Felinum, Lac Vaccinum Defloratum, Lachesis, Lachnanthes, Lactuca Virosa, Lamium, Lapathum, Latrodectus Mactans, Lecithinum, Ledum Palustre, Leptandra, Lilium Tigrinum, Limulus Cyclops, Lithium Carbonicum, Lobelia Cardinalis, Lobelia Dortmanna, Lobelia Inflata, Lobelia Purpurascens, Loleum Temulentum, Lonicera Xylosteum, Luna, Lupulus Humulus, Lycopersicum, Lycopodium Clavatum, Lycopus, Lyssinum, Macrotinum, Magnesia Muriatica, Magnesia Phosphorica, Magnesium Sulphuricum, Mancinella, Manganum, Medorrhinum, Melilotus Officinalis, Menispermum, Mentha Piperita, Mentha Pulegium, Mentholum, Menyanthes, Mercurialis Perennis, Mercurius Corrosivus, Mercurius Cyanatus, Mercurius Solubilis, Mezereum, Millefolium, Momordica Balsamica, Morphinum, Moschus, Muriaticum Acidum, Myrica Cerifera, Naja Tripudians, Natrium Arsenicosum, Natrium Nitricum, Natrium Phosphoricum, Natrium Salicylicum, Natrum Carbonicum, Natrum Hypochlorosum, Natrum Muriaticum, Natrum Sulphuricum, Niccolum Metallicum, Niccolum Sulphuricum, Nitricum Acidum, Nuphar Luteum, Nux Moschata, Nux Vomica, Nyctanthes Arbor Tristis, Oenanthe Crocata, Oleander, Oleum Animale Aethereum, Oleum Jecoris Aselli, Oniscus Asellus, Ononis, Onosmodium Virginianum, Opium, Oreodaphne, Osmium, Ostrya, Oxalicum Acidum, Oxytropis Lamberti, Paeonia, Palladium, Paraffinum, Paris, Paris Quadrifolia, Parthenium, Passiflora Incarnata, Penthorum Sedoides, Petroleum, Phaseolus, Phellandrium, Phenacetinum, Phosphoricum Acidum, Phosphorus, Physalis Alkekengi, Physostigma, Physostigma Venenosum, Phytolacca, Picricum Acidum, Pimpinella, Piper Methysticum, Piper Nigrum, Plantago Major, Platinum Metallicum, Plumbum Metallicum, Podophyllum, Polyporus, Populus Candicans, Pothos Foetida, Primula Veris, Prunus Padus, Prunus Spinosa, Psorinum, Ptelea,. Pulex Irritans, Pulsatilla Pratensis, Pulsatilla, Pyrogenium, Radium Bromatum, Ranunculus Bulbosus, Ranunculus Glacialis, Ranunculus Sceleratus, Raphanus, Ratanhia Peruviana, Rhamnus Californica, Rheum, Rhodium Oxydatum Nitricum, Rhododendron Ferrugineum, Rhus Glabra, Rhus Toxicodendron, Rhus Venenata, Ricinus Communis, Robinia, Ruta Graveolens, Sabadilla, Sabal Serrulata, Sabina, Saccharum Lactis, Saccharum Officinale, Salicylicum Acidum, Salix Alba, Salol, Sambucus Nigra, Sanguinaria Canadensis, Sanguinarinum Nitricum, Sanicula, Santoninum, Saponaria Officinalis, Saponinum, Sarsaparilla, Scrophularia Nodosa, Scutellaria Laterifolia, Secale Cornutum, Selenium, Senecio Aureus, Senecio Jacoboea, Senega, Sepia Officinalis, Silica, Silicea Terra, Sinapis Alba, Sinapis Nigra, Skatolum, Sol, Solanum Nigrum, Solanum Tuberosum Egrotans, Spigelia Anthelmia, Spigelia Marilandica, Spongia Tosta, Stachys Betonica, Stannum, Staphysagria, Stellaria Media, Sticta Pulmonaria, Stillingia Sylvatica, Stramonium, Strontium Carbonicum, Strontium Nitricum, Strophanthus Hispidus, Strychninum, Succinum, Sulfonal, Sulphur, Sulphuricum Acidum, Sulphurosum Acidum, Sumbulus Moschatus, Symphytum Officinale, Syphilinum, Tabacum, Tanacetum Vulgare, Taraxacum Officinale, Tarentula Cubensis, Tarentula Hispanica, Taxus Baccata, Tellurium Metallicum, Terebinthiniae Oleum, Teucrium Marum Verum, Thea Chinensis, Theridion, Thlaspi Bursa Pastoris, Thuja Occidentalis, Thyroidinum, Tilia Europaea, Tongo, Torula Cerevisiae, Trifolium Pratense, Trillium Pendulum, Trinitrotoluenum, Triosteum Perfoliatum, Tuberculinum, Turnera Aphrodisiaca, Tussilago Petasites, Upas, Upas Tieut, Uranium Nitricum, Urtica Urens, Usnea Barbata, Ustilago, Ustilago Maydis, Vaccininum, Valeriana, Variolinum, Veratrum Album, Veratrum Nigrum, Veratrum Viride, Verbascum Thapsus, Viburnum Opulus, Vinca Minor, Viola Odorata, Viola Tricolor, Viscum Album, Wyethia, Xanthoxylum, Xerophyllum, X-Ray, Yohimbinum, Yucca Filamentosa, Zincum, Zincum Metallicum, Zincum Phosphoricum, Zincum Picricum, Zincum Sulphuricum, Zincum Valerianicum, Zingiber Officinale, Zizia, and any combination thereof.

In accordance with a further embodiment of the present invention there is provided a kit for treating, ameliorating or at least reducing pain intensity associated with a headache in a host suffering therefrom, the kit comprising

a container comprising a topical formulation to be applied to the cephalic region of said host, said topical formulation comprising a therapeutically effective amount of an active agent being selected from the group consisting of: Absinthium, Aceticum Acidum, Actaea Spicata, Agaricus Muscarius, Aceticum Acid, Aconitum Cammarum, Aconitum Napellus, Aesculus Hippocastanum, Aethusa, Ailanthus Glandulosa, Alfalfa, Allium Cepa, Allium Sativum, Aloe Socotrina, Alumen, Alumina, Ambra Grisea, Ammoniacum Gummi, Ammonium lodatum, Ammonium Picricum, Amphisbaena, Amygdalae Amarae Aqua, Amylenum Nitrosum, Anacardium Orientale, Anagallis Arvensis, Angustura Vera, Anhalonium Lewinii, Anthemis Nobilis, Antimonium Crudum, Antimonium Tartaricum, Antipyrinum, Aphis Chenopodii Glauci, Apis Mellifica, Apium Graveolens, Apocynum Cannabinum, Apomorphinum, Aqua Marina, Aranea Diadema, Aranea Scinencia, Argemone Mexicana, Argentum Metallicum. Argentum Nitricum, Armoracea Sativa, Arnica, Arsenicum Album, Arsenicum Metallicum, Arsenicum Sulphuratum Flavum, Arum Dracontium, Arum Italicum, Arum Triphyllum, Arundo Mauritanica, Asa Foetida, Asarum Europaeum, Asclepias Cornuti, Asclepias Syriaca, Asclepias Tuberosa, Asparagus Officinalis, Asterias Rubens, Astragalus Menziesii, Astragalus Mollissimus, Athamanta, Aurantium, Aurum Arsenicicum, Aurum Bromatum, Aurum Metallicum, Aurum Muriaticum Natronatum, Avena Sativa, Azadirachta Indica, Bacillinum Burnett, Badiaga, Baptisia Confusa Acetica, Baptisia Tinctoria, ryta Carbonica, Baryta Muriatica, Belladonna, Bellis Perennis, Benzinum, Benzoicum Acidum, Benzolum, Berberis Aquifolium, Berberis Vulgaris, Bismuthum Subnitricum, Boletus Laricis, Boracicum Acidum, Borax Veneta, Bovista Lycoperdon, Bromium, Brucea Antidysenterica, Brucinum, Bryonia Alba, Bufo Rana, Butyricum Acidum, Cactus Grandiflorus, Cadmium Sulphuratum, Cajuputum, Caladium Seguinum, Calcarea Acetica, Calcarea Arsenicosa, Calcarea Carbonica, Calcarea-Fluorica, Calcarea Hypophosphorosa, Calcarea lodata, Calcarea Lactica, Calcarea Phosphorica, Calendula Officinalis, Camphora, Cannabis Indica, Cantharis Vesicatoria, Capsicum Annuum, Carbo Animalis, Carbo Vegetabilis, Carbolicum Acidum, Carboneum Oxygenisatum, Carboneum Sulphuratum, Carduus Benedictus, Carduus Marianus, Carlsbad Aqua, Cascara Sagrada, Causticum, Cedron, Cenchris Contortrix, Cereus Bonplandii, Chamomilla, Chelidonium Majus, Chenopodium Anthelminticum, Chimaphila Maculata, Chimaphila Umbellata, China Officinalis, Chininum Arsenicosum, Chininum Muriaticum, Chininum Sulphuricum, Chionanthus Virginica, Chloralum Hydratum, Cichorium, Cicuta Virosa, Cimex Lectularius, Cimicifuga Racemosa, Cina Maritima, Cinchona Off, Cinchoninum Sulphuricum, Cinnamomum, Clematis Erecta, Clematis Erecta, Cobaltum Metallicum, Coca-Erythroxylon Coca, Cocainum Hydrochloricum, Coccinella Septempunctata, Cocculus Indicus, Coccus Cacti, Cochlearia Armoracia, Codeinum, Coffea Cruda, Colchicum Autumnale, Collinsonia Canadensis, Colocynthinum, Colocynthis, Condurango, Conium Maculatum, Convallaria Majalis, Convolvulus Duartinus, Copaiva Officinalis, Corallium Rubrum, Cornus Circinata, Crataegus Oxyacantha, Crocus Sativus, Crotalus Cascavella, Crotalus Horridus, Croton Tiglium, Cundurango, Cupressus Australis, Cuprum Aceticum, Cuprum Metallicum, Curare, Cyclamen Europaeum, Cypripedium Pubescens, Daphne Indica, Digitalis Purpurea, Dioscorea Villosa, Dirca Palustris, Drosera Rotundifolia, Dulcamara, Echinacea Angustifolia, Elaeis Guineensis, Elaps Corallinus, Electricitas, Ephedra Vulgaris, Epiphegus Virginiana, Eserinum, Eucalyptus Globulus, Eugenia Jambos, Euonymus Atropurpurea, Euonymus Europaea, Eupatorium Perfoliatum, Eupatorium Purpureum, Euphorbia Hypericifolia, Euphorbium Officinarum, Euphrasia Officinalis, Eupionum, Fagopyrum Esculentum, Fagus, Fel Tauri, Ferrum Bromatum, Ferrum Metallicum, Ferrum Muriaticum, Ferrum Phosphoricum, Ferrum Sulphuricum, Ferrum Tartaricum, Feverfew, Ficus Religiosa, Fluoricum Acidum, Formica Rufa, Franciscea Uniflora, Fraxinus Americana, Gallicum Acidum, Gaultheria Procumbens, Gelesemium Sempervirens, Gelsemium Sempervirens, Genista, Gentiana Lutea, Geranium Maculatum, Ginger, Ginseng, Ginseng Quinquefolium, Glonoine, Glonoinum, Glycerinum, Gnaphalium Polycephalum, Goldenseal, Gossypium Herbaceum, Graphites, Gratiola, Gratiola Officinalis, Grindelia Robusta, Guaco, Guaiacum, Guajacum Officinale, Guarana, Guarea Trichiloides, Gymnocladus Canadensis, Haematoxylon, Haematoxylon Campechianum, Hamamelis Virginiana, Hedeoma Pulegioides, Helleborus Niger, Heloderma, Helonias Dioica, Hepar Sulphur, Heracleum Sphondylium, Herbs Dandelion, Hippuricum Acidum, Homarus, Hydrastis Canadensis, Hydrocyanicum Acidum, Hydrophobinum, Hyoscyamus Niger, Hypericum, Hypericum Perforatum, Iberis Amara, lchthyolum, Ictodes Foetida, Ignatia Amara, Indigo Tinctoria, Indium Metallicum, Indolum, Inula Helenium, lodium, lodoformum, Ipecacuanha, Iridium Metallicum, Iris Florentina, Iris Fcetidissima, Iris Tenax, Iris Versicolor, Jacaranda Caroba, Joanesia Asoca, Juglans Cinerea, Juglans Regia, Juncus, Justicia Adhatoda, Kali Carbonicum, Kali Cyanatum, Kali Nitricum, Kali Oxalicum, Kali Phosphoricum, Kalium Bromatum, Kalium lodatum, Kalium Muriaticum, Kalium Silicicum, Kalium Sulphuricum, Kalmia Latifolia, Kreosotum, Lac Caninum, Lac Felinum, Lac Vaccinum Defloratum, Lachesis, Lachnanthes, Lactuca Virosa, Lamium, Lapathum, Latrodectus Mactans, Lecithinum, Ledum Palustre, Leptandra, Lilium Tigrinum, Limulus Cyclops, Lithium Carbonicum, Lobelia Cardinalis, Lobelia Dortmanna, Lobelia Inflata, Lobelia Purpurascens, Loleum Temulentum, Lonicera Xylosteum, Luna, Lupulus Humulus, Lycopersicum, Lycopodium Clavatum, Lycopus, Lyssinum, Macrotinum, Magnesia Muriatica, Magnesia Phosphorica, Magnesium Sulphuricum, Mancinella, Manganum, Medorrhinum, Melilotus Officinalis, Menispermum, Mentha Piperita, Mentha Pulegium, Mentholum, Menyanthes, Mercurialis Perennis, Mercurius Corrosivus, Mercurius Cyanatus, Mercurius Solubilis, Mezereum, Millefolium, Momordica Balsamica, Morphinum, Moschus, Muriaticum Acidum, Myrica Cerifera, Naja Tripudians, Natrium Arsenicosum, Natrium Nitricum, Natrium Phosphoricum, Natrium Salicylicum, Natrum Carbonicum, Natrum Hypochlorosum, Natrum Muriaticum, Natrum Sulphuricum, Niccolum Metallicum, Niccolum Sulphuricum, Nitricum Acidum, Nuphar Luteum, Nux Moschata, Nux Vomica, Nyctanthes Arbor Tristis, Oenanthe Crocata, Oleander, Oleum Animale Aethereum, Oleum Jecoris Aselli, Oniscus Asellus, Ononis, Onosmodium Virginianum, Opium, Oreodaphne, Osmium, Ostrya, Oxalicum Acidum, Oxytropis Lamberti, Paeonia, Palladium, Paraffinum, Paris, Paris Quadrifolia, Parthenium, Passiflora Incarnata, Penthorum Sedoides, Petroleum, Phaseolus, Phellandrium, Phenacetinum, Phosphoricum Acidum, Phosphorus, Physalis Alkekengi, Physostigma, Physostigma Venenosum, Phytolacca, Picricum Acidum, Pimpinella, Piper Methysticum, Piper Nigrum, Plantago Major, Platinum Metallicum, Plumbum Metallicum, Podophyllum, Polyporus, Populus Candicans, Pothos Foetida, Primula Veris, Prunus Padus, Prunus Spinosa, Psorinum, Ptelea, Pulex Irritans, Pulsatilla Pratensis, Pulsatilla, Pyrogenium, Radium Bromatum, Ranunculus Bulbosus, Ranunculus Glacialis, Ranunculus Sceleratus, Raphanus, Ratanhia Peruviana, Rhamnus Californica, Rheum, Rhodium Oxydatum Nitricum, Rhododendron Ferrugineum, Rhus Glabra, Rhus Toxicodendron, Rhus Venenata, Ricinus Communis, Robinia, Ruta Graveolens, Sabadilla, Sabal Serrulata, Sabina, Saccharum Lactis, Saccharum Officinale, Salicylicum Acidum, Salix Alba, Salol, Sambucus Nigra, Sanguinaria Canadensis, Sanguinarinum Nitricum, Sanicula, Santoninum, Saponaria Officinalis, Saponinum, Sarsaparilla, Scrophularia Nodosa, Scutellaria Laterifolia, Secale Cornutum, Selenium, Senecio Aureus, Senecio Jacoboea, Senega, Sepia Officinalis, Silica, Silicea Terra, Sinapis Alba, Sinapis Nigra, Skatolum, Sol, Solanum Nigrum, Solanum Tuberosum kgrotans, Spigelia Anthelmia, Spigelia Marilandica, Spongia Tosta, Stachys Betonica, Stannum, Staphysagria, Stellaria Media, Sticta Pulmonaria, Stillingia Sylvatica, Stramonium, Strontium Carbonicum, Strontium Nitricum, Strophanthus Hispidus, Strychninum, Succinum, Sulfonal, Sulphur, Sulphuricum Acidum, Sulphurosum Acidum, Sumbulus Moschatus, Symphytum Officinale, Syphilinum, Tabacum, Tanacetum Vulgare, Taraxacum Officinale, Tarentula Cubensis, Tarentula Hispanica, Taxus Baccata, Tellurium Metallicum, Terebinthiniae Oleum, Teucrium Marum Verum, Thea Chinensis, Theridion, Thlaspi Bursa Pastoris, Thuja Occidentalis, Thyroidinum, Tilia Europaea, Tongo, Torula Cerevisiae, Trifolium Pratense, Trillium Pendulum, Trinitrotoluenum, Triosteum Perfoliatum, Tuberculinum, Turnera Aphrodisiaca, Tussilago Petasites, Upas, Upas Tieut, Uranium Nitricum, Urtica Urens, Usnea Barbata, Ustilago, Ustilago Maydis, Vaccininum, Valeriana, Variolinum, Veratrum Album, Veratrum Nigrum, Veratrum Viride, Verbascum Thapsus, Viburnum Opulus, Vinca Minor, Viola Odorata, Viola Tricolor, Viscum Album, Wyethia, Xanthoxylum, Xerophyllum, X-Ray, Yohimbinum, Yucca Filamentosa, Zincum, Zincum Metallicum, Zincum Phosphoricum, Zincum Picricum, Zincum Sulphuricum, Zincum Valerianicum, Zingiber Officinale, Zizia, and any combination thereof.

In an embodiment, the active agent or agents of the invention have been diluted so as extract their active medicinal properties and to reach their therapeutically effective potency DEFINITIONS

The term “headache” should be construed to also include, without limitation, a tension headache, a migraine, a sinus headache, a cluster headache, and a rebound headache. Headaches are a common problem and are usually not a sign of a serious medical condition. Most headaches are minor. Headaches can be caused by a number of different factors such as stress/anxiety, alcohol, atmosphere, lighting, sitting at a computer for long periods of time, allergies, eye strain, low blood sugar, grinding teeth, poor posture, sleep loss etc. The pains are described as “a weight bearing down on the head”, as “a tight, pinching headband” or as “a dull, empty feeling”. Many researchers believe that a tension headache can result when the head, neck, or face muscles contract but do not relax. They remain tense or in a spasm. This compresses blood vessels in the affected muscles, and reduces the blood supply there. The resulting pain feels like a squeezing, aching pressure at the temples, across the upper forehead, in the face, or in the back of the neck. Tension headaches may last for hours, sometimes days, and are usually mildly to moderately painful. More than 45 million Americans suffer from chronic headaches and of them 28 million suffer from migraines. Most headaches are not migraine. Some known headache triggers include: diet, eating and sleeping patterns, emotions (such as depression, stress, anxiety, frustration, letdown), medications, environment among other triggers. For women, one main trigger is a change in the body's level of hormones.

The expression: “a homeopathic ingredient known for treating, ameliorating or at least reducing pain intensity associated with a headache in a host suffering therefrom when administered internally” should be construed herein to comprise all homeopathic drugs listed in the Homeopathic Pharmacopoeia of the United States (HPUS) or other competent and recognized Pharmacopeia known for treating, ameliorating or at least reducing pain intensity associated with a headache when taken internally. The Homeopathic Pharmacopoeia of the United States (HPUS) is the official compendium for Homeopathic Drugs in the U.S. The HPUS is declared a legal source of information on drug products (along with the USP/NF) in the Federal Food Drug and Cosmetic Act, 21 U.S.C. § 301. Section 201(g)(1) of the Act. 21 U.S.C. § 321 defines the term “drug” as “articles recognized in the official United States Pharmacopoeia, official Homceopathic Pharmacopoeia of the United States, or official National Formulary or any supplement to any of them. The following authorities document homeopathic provings that evidence the efficacy of certain homeopathic ingredients included in the HPUS in treating the symptoms associated with headaches and migraines in those suffering therefrom: Allen T. P., Encyclopedia of pure materia medica, 10 vol., Edit. Boericke et Tafel, New-York, 1879-1974; Boericke, W., Pocket manual of homeopathic materia medica with repertory; Boericke and Tafel, The Family Guide to Self-Medication; Callinan, Paul, M. D., Family Homeopathy—A Practical Guide for Home Treatment; Clarke, J. H., A Dictionary of Practical Materia Medica, 3 vol., The Hom. Publ. Co., London, 1902; Farrington, E. A., Clinical Materia Medica; Gentry, W. D., Concordance repertory, 6 vol; Hahnemann, S., Materia medica pura, 2 vol; Hering C., The guiding symptoms of our materia medica, 10 vol. Edit; Boericke et Tafel, Philadelphia, 1890; Kent, J. T., Repertory of homeopathic materia medica; Lockie, Andrew, M. D., The Family Guide to Homeopathy Symptoms & Natural Solutions; LECTURES ON HOMOEOPATHIC MATERIA MEDICA by JAMES TYLER KENT, A. M., M.D.; HOMCEOPATHIC MATERIA MEDICA by William BOERICKE, M.D.; Murphy, R., New homeopathic medical repertory; Vithoulkas, G., Materia Medica Viva, volume 1. The foregoing are incorporated herein by reference. More specifically, the provings found in these authorities demonstrate the efficacy of said homeopathic ingredients in treating headaches and migraines when taken internally.

“Cluster headaches” are the rarest class of headache, affecting less than 1% of the population. Attacks occur daily in clusters of weeks or months, then disappear completely for months or years. 90% of sufferers are males, most aged 20-30.

“Sinus Headache”: sinus pain is the result of pressure behind the eyes, along the nose, and behind the cheeks. Sometimes the pressure can feel no more serious than a stuffy nose. In other cases the pain can be intense enough to keep one from daily routines. Sinus headaches are relatively uncommon, contrary to popular belief. They are often caused by Sinusitis. Most people who think they have sinus headaches actually suffer from tension headaches or migraines.

“Rebound headaches” occur in patients whose persistent headache pain causes them to overuse headache medication. This may cause symptoms to worsen instead of improve.

Throughout this application, the term “about” is used to indicate that a value includes the standard deviation of error for the device or method being employed to determine the value. In an embodiment, this deviation is ±5% of a given value.

The term “pharmaceutically acceptable carrier” or “pharmaceutically acceptable vehicle” or “excipient” should be construed herein to include materials that are suitable for topical administration to the head region that are not biologically or otherwise undesirable, i.e., that may be administered to an individual along with an active agent without causing any undesirable biological effects. Moreover, the terms “carriers”, “diluents”, “excipients” and “pharmaceutically acceptable salt” are to be construed as defined in the reference manual: “Handbook of Pharmaceutical Excipients” third edition, Arthur H. Kibbe, American Pharmaceutical Association, Pharmaceutical Press, and incorporated by reference herein.

The term “cooling agent” should be construed to include compounds whose action on thermoreceptors provides a cooling sensation via cold receptors. The following references list examples of possible cooling agents: Watson, H. R., “Flavor Characteristics Of Synthetic Cooling Compounds” in Flavor: It's Chemical, Behavioral & Commercial Aspects; C. M. Apt, Ed., Westview Press, Boulder, 1977, pp. 31-50; Watson, H. R., R. Hems, D. G. Roswell & D. J. Spring, New Compounds with the Menthol Cooling Effect, J. Soc. Cosmet. Chem., 29,185-200 (1978); Roswell, David G.; Spring, David J.; Hems, Roger, Acyclic carboxamides having a physiological cooling effect, U.S. Pat. No. 4,296,255, Oct. 20, 1981, Assigned to Wilkinson Sword Limited; Rowsell, David G.; Gascoyne, John M.; Hems, Roger, Cyclic carboxamides having a physiological cooling effect , U.S. Pat. No. 4,296,093, Oct. 20, 1981, Assigned to Wilkinson Sword Limited; Roswell, David G.; Spring, David J.; Hems, Roger, Acyclic carboxamides having a physiological cooling effect, U.S. Pat. No. 4,230,688, Oct. 28, 1980, Assigned to Wilkinson Sword Limited; Watson, Hugh R.; Rowsell, David G.; Spring, David J., N-substituted paramenthane carboxamides, U.S. Pat. No. 4,226,988, Oct. 7, 1980, Assigned to Wilkinson Sword Limited; Watson, Hugh R.; Rowsell, David G.; Spring, David J., N-substituted paramenthane carboxamides, U.S. Pat. No. 4,193,936, Mar. 18, 1980, Assigned to Wilkinson Sword Limited; Watson, Hugh R.; Rowsell, David G.; Spring, David J., N-substituted paramenthane carboxamides, U.S. Pat. No. 4,178,459, Dec. 11, 1979, Assigned to Wilkinson Sword Limited; Roswell, David G.; Spring, David J.; Hems, Roger, Acyclic carboxamides having a physiological cooling effect, U.S. Pat. No. 4,153,679, May 8, 1979, Assigned to Wilkinson Sword Limited; Watson, Hugh R.; Rowsell, David G.; Spring, David J., N-substituted paramenthane carboxamides, U.S. Pat. No. 4,150,052, Apr. 17, 1979, Assigned to Wilkinson Sword Limited; Rowsell, David G.; Spring, David J., Phosphine oxides having a physiological cooling effect, U.S. Pat. No. 4,070,496, Jan. 24, 1978, Assigned to Wilkinson Sword Limited; Rowsell, David G.; Hems, Roger, Compounds having a physiological cooling effect and compositions containing them, U.S. Pat. No. 4,070,449, Jan. 24, 1978, Assigned to Wilkinson Sword Limited; Watson, Hugh R.; Rowsell, David G.; Spring, David J., Tobacco and tobacco-containing manufactures containing an ingredient having physiological cooling activity, U.S. Pat. No. 4,060,091, Nov. 29,1977, Assigned to Wilkinson Sword Limited; Watson, Hugh R.; Rowsell, David G.; Browning, John H. D., Tobacco and tobacco-containing manufactures containing an ingredient having physiological cooling activity, U.S. Pat. No. 4,059,118, Nov. 22, 1977, Assigned to Wilkinson Sword Limited; Rowsell, David G.; Hems, Roger, Compounds having a physiological cooling effect and compositions containing them, U.S. Pat. No. 4,034,109, Jul. 5, 1977, Assigned to Wilkinson Sword Limited; Watson, Hugh R.; Rowsell, David G.; Browning, John H. D., Substituted p-menthanes, U.S. Pat. No. 4,033,994, Jul. 5, 1977, Assigned to Wilkinson Sword Limited; Rowsell, David G.; Spring, David J., Cyclic sulphoxides and sulphones having a physiological cooling action on the human body, U.S. Pat. No. 4,032,661, Jun. 28, 1977, Assigned to Wilkinson Sword Limited; Rowsell, David G.; Spring, David J., Cyclic sulphonamides and sulphinamides having a physiological cooling effect, U.S. Pat. No. 4,020,153, Apr. 26, 1977, Assigned to Wilkinson Sword Limited; Grub, Helmut; Pelzer, Ralf; Hopp, Rudolf; Emberger, Roland; Bertram; Heinz-Jurgen, Compositions which have a physiological cooling effect, and active compounds suitable for these compositions, U.S. Pat. No. 5,266,592, Nov. 30, 1993, Assigned to Haarmann & Reimer GmbH; Amano, Akira; Moroe, Michio; Yoshida, Toshio, 3-Levo-Menthoxypropane-1,2-diol, U.S. Pat. No. 4,459,425, Jul. 10, 1984. Assigned to Takasago Perfumery Co., Ltd; Yamamoto, Takeshi; Method for purifying (−)-N-isopulegol and citrus perfume composition containing (−)-N-isopulegol obtained by the method, U.S. Pat. No. 5,773,410, Jun. 30, 1998. Assigned to Takasago International Corporation; Suares; Alan Joseph; Znaiden; Alexander Paul; Feliciano; Donald Carl; Carrabotta; Michele, Cosmetic compositions with sensate mixtures based on isopulegol, U.S. Pat. No. 6,267,974, Jul. 31, 2001. Assigned to Unilever Home & Personal Care USA, Division of Conopco, Inc; Ottinger, H., T. Soldo & T. Hoffmann, Systematic Studies on Structure and Physiological Activity of Cyclic alpha-Keto Enamines, A Novel Class of “Cooling Compounds”, J. Agric. Food Chem., 49, 5383-5390 (2001). See also Hofmann, Thomas; Ottinger, Harald; Frank, Oliver; Soldo, Tomislav; Cerny, Christoph; Robert, Fabien; Blank; Imre; Method of using alpha-keto enamine derivatives as ingredients and products incorporating same, U.S. Pat. No. 6,592,884, Jul. 15, 2003; Kenmochi; Hiroyuki; Akiyama; Teruyoshi; Yuasa; Yoshifumi; Kobayashi; Toyohiko; Tachikawa; Akio, Method for producing para-menthane-3,8-diol, U.S. Pat. No. 5,959,161, Sep. 28, 1999. Assigned to Takasago International Corporation; Shiroyama; Kenichiro; Sawano; Kiyohito; Ohta; Hideaki , Cool feeling composition, U.S. Pat. No. 6,328,982, Dec. 11, 2001. Assigned to Takasago International Corporation; Frerot; Eric & Van Beem; Nicole, Compounds derived from menthol and use as refreshing agent, U.S. Pat. No. 6,359,168, Mar. 19, 2002 . Assigned to Firmenich S A; Velazco; Maria Ines; Wuensche; Laurent; Deladoey; Patrice, Use of cubebol as a flavoring ingredient, U.S. Pat. No. 6,214,788, Apr. 10, 2001. Assigned to Firmenich S A; Amano; Akira, Tokoro; Kazuhiko, (2S)-3-[(1R, 2S, 5R)-[5-methyl-2-(1-methylethyl)-cyclohexyl]oxy]-1,2-propanediol, process for producing the same, and compositions containing the same, U.S. Pat. No. 5,608,119, Mar. 4, 1997. Assigned to Takasago International Corporation; TAKAZAWA OSAMU; WATANABE HIROYUKI; ISO MASAKI , p-MENTHANE DERIVATIVE AND COLD SENSING AGENT CONTAINING THE SAME, Japanese Patent No. 2004059474, Feb. 26, 2004. Assigned to T. Hasegawa Co., Ltd; Foster, Alison Jane; Van Der Logt, Cornelius Paul Erik; Tareilus, Erwin Werner; Novel compounds and their uses, U.S. Patent Application 20040067970, Apr. 8, 2004. The foregoing are incorporated herein by reference.

The term “therapeutically effective amount” should be construed herein to include a nontoxic but sufficient amount of the agent or ingredient to provide the desired therapeutic effect. The exact amount required will vary from subject to subject, depending on the age, weight, and general condition of the subject, the severity of the condition being treated, and the like. Thus, it is not possible to specify an exact “effective amount.” However, an appropriate “effective amount” in any individual case may be determined by one of ordinary skill in the art using only routine experimentation.

The terms “ingredient”, “carrier” and “agent” should be construed to include both the singular and the plural forms thereof.

The term “host” should be construed herein to include humans as well as other mammals

“Homeopathy” or “Homeopathic medicine” is a natural pharmaceutical science that uses various plants, minerals or animals in very small doses to stimulate the sick person's natural defenses. “Homoios” in Greek means similar and “pathos” means disease or suffering. Since one's symptoms are actually efforts of the organism to reestablish homeostasis or balance, it is logical to seek a substance that would, in overdose, cause the similar symptoms the person is experiencing. The medicines, thus, go with, rather than against, the person's natural defenses. The homeopath seeks to find a substance that would cause the similar symptoms the person has and then gives it in small, specially prepared doses. The potencies of homeopathic drugs are specified in terms of dilution. Homeopathic medicines are made by taking an original susbstance, which may be plants, minerals, animal organs, and other substances, and diluting them according to the principles and guidelines found in the HPUS. They may be diluted in water or alcohol if they will dissolve, or lactose (milk sugar) powder if they won't dissolve. They may be diluted to one part in 10 or one part in 100. These would be called 1X or 1C by homeopaths, using the Roman numerals X=10 and C=100 for the magnitude of dilution. The solution or powder is then shaken. A part of it is taken and diluted again, by the same factor as before. The concentration of the original substance would now be one part in 100 for 2X or one part in 10,000 for 2C. The dilutions are repeated again and again. The table below summarises some of the dilutions applied to common homeopathic medicines. Due to the fact that the more diluted the active agent is, the more potent it is, these dilutions are referred to as ‘potencies’.

| 1X: 1 part in 10 | 1C: 1 part in 100 |
|---|---|
| 2X: 1 part in 100 | 2C: 1 part in 10,000 |
| 3X: 1 part in 1000 | 3C: 1 part in 1,000,000 |
| 4X: 1 part in 10,000 | 4C: 1 part in 100,000,000 |
| 5X: 1 part in 100,000 | 5C: 1 part in 10,000,000,000 |
| 6X: 1 part in 1,000,000 | 10C: 1 part in 100,000,000,000,000,000,000 |
| 10X: 1 part in 10,000,000,000 | 20C: 1 part in 10,000,000,000,000,000,000,000,000,000,000,000,000,000 |
| 20X: 1 part in 100,000,000,000,000,000,000 | 30C: 1 part in |
| | 1,000,000,000,000,000,000,000,000,000,000,000,000,000,000,000,000,000,000,000,000 |

The term “trituration” or “dry attenuation” should be construed herein as the grinding of substances such as plants, herbs and powders using a mortar and pestle for example or with other suitable devices. It is the primary mode of mixing the ingredients used in preparation of powdered dilutions in homeopathy.

The term “Potency” should be construed herein to refer to the concentration of the active agent after attenuation with a diluent. For example, mixing 1 part of an active agent with 9 parts diluent yields a 1X Potency, which refers to 1/10th of the original concentration. Mixing 1 part of this 1X Potency with 9 parts diluent yields 2X Potency, which refers to 1/100th of the original concentration. Mixing 1 part of this 2X Potency with 9 parts diluent yields 3X Potency, which refers to 1/1000th of the original concentration. This process is repeated until the desired potency is attained.

The terms “active agent(s)”, “active ingredient(s)”, “homeopathic ingredient(s)”, “homeopathic drug(s)”, “homeopathic agent(s)”, “homeopathic active agent(s)”, “homeopathic active ingredient(s)” and “drug(s)” should all be construed herein to comprise those substances recognized and listed in the official compendium for Homeopathic Drugs in the U.S., the HPUS, or recognized and listed in another offically recognized pharmacopeia, such as the French or German Pharmacopeias:Homöopathische Arzneimittel (German Homeopathic Pharmacopeia) (HAB), Pharmacopée franicaise (PhF).

When referring to dilutions from about 1X to about 200C, all increments therebetween are also included.

Other objects, advantages and features of the present invention will become more apparent upon reading of the following non restrictive description of embodiments thereof, given by way of example only.

### DESCRIPTION OF EMBODIMENTS

Embodiments of the invention will be herein described so as to exemplify the invention and not limit the scope thereof.

The present invention provides for homeopathic ingredients that have been proven useful in treating headaches when taken internally, to be used for methods, formulations and kits that provide for such ingredients to be applied topically. The present invention also provides for easy-to-use kits in order to treat the symptoms described herein.

The present invention provides methods, topical formulations and kits useful in treating a host suffering from a headache, i.e., from a pain in the head, cephalalgia, headache pain. The headache may be any of a variety of different types of headaches, including but not limited to: tension headache, migraine headache, cluster headache, headache associated with inflamed sinuses, or other causes.

In accordance with the present invention, the homeopathic active agents disclosed herein ameliorate, treat (which includes stimulating the body's curative powers to overcome symptoms) or at least reduce the pain associated with a headache when topically applied to the cephalic (or head) region of a host suffering therefrom. Hence, the present invention provides a topical homeopathic method, formulations and kits for headache therapy.

The active agents of the invention comprise a homeopathic drug or drugs known for treating, ameliorating or at least reducing pain intensity associated with a headache in a host suffering therefrom when administered internally.

In an embodiment, the active agent of the invention is selected from the group consisting of: Absinthium, Aceticum Acidum, Actaea Spicata, Agaricus Muscarius, Aceticum Acid, Aconitum Cammarum, Aconitum Napellus, Aesculus Hippocastanum, Aethusa, Ailanthus Glandulosa, Alfalfa, Allium Cepa, Allium Sativum, Aloe Socotrina, Alumen, Alumina, Ambra Grisea, Ammoniacum Gummi, Ammonium lodatum, Ammonium Picricum, Amphisbaena, Amygdalae Amarae Aqua, Amylenum Nitrosum, Anacardium Orientale, Anagallis Arvensis, Angustura Vera, Anhalonium Lewinii, Anthemis Nobilis, Antimonium Crudum, Antimonium Tartaricum, Antipyrinum, Aphis Chenopodii Glauci, Apis Mellifica, Apium Graveolens, Apocynum Cannabinum, Apomorphinum, Aqua Marina, Aranea Diadema, Aranea Scinencia, Argemone Mexicana, Argentum Metallicum. Argentum Nitricum, Armoracea Sativa, Arnica, Arsenicum Album, Arsenicum Metallicum, Arsenicum Sulphuratum Flavum, Arum Dracontium, Arum Italicum, Arum Triphyllum, Arundo Mauritanica, Asa Foetida, Asarum Europaeum, Asclepias Cornuti, Asclepias Syriaca, Asclepias Tuberosa, Asparagus Officinalis, Asterias Rubens, Astragalus Menziesii, Astragalus Mollissimus, Athamanta, Aurantium, Aurum Arsenicicum, Aurum Bromatum, Aurum Metallicum, Aurum Muriaticum Natronatum, Avena Sativa, Azadirachta Indica, Bacillinum Burnett, Badiaga, Baptisia Confusa Acetica, Baptisia Tinctoria, ryta Carbonica, Baryta Muriatica, Belladonna, Bellis Perennis, Benzinum, Benzoicum Acidum, Benzolum, Berberis Aquifolium, Berberis Vulgaris, Bismuthum Subnitricum, Boletus Laricis, Boracicum Acidum, Borax Veneta, Bovista Lycoperdon, Bromium, Brucea Antidysenterica, Brucinum, Bryonia Alba, Bufo Rana, Butyricum Acidum, Cactus Grandiflorus, Cadmium Sulphuratum, Cajuputum, Caladium Seguinum, Calcarea Acetica, Calcarea Arsenicosa, Calcarea Carbonica, Calcarea Fluorica, Calcarea Hypophosphorosa, Calcarea lodata, Calcarea Lactica, Calcarea Phosphorica, Calendula Officinalis, Camphora, Cannabis Indica, Cantharis Vesicatoria, Capsicum Annuum, Carbo Animalis, Carbo Vegetabilis, Carbolicum Acidum, Carboneum Oxygenisatum, Carboneum Sulphuratum, Carduus Benedictus, Carduus Marianus, Carlsbad Aqua, Cascara Sagrada, Causticum, Cedron, Cenchris Contortrix, Cereus Bonplandii, Chamomilla, Chelidonium Majus, Chenopodium Anthelminticum, Chimaphila Maculata, Chimaphila Umbellata, China Officinalis, Chininum Arsenicosum, Chininum Muriaticum, Chininum Sulphuricum, Chionanthus Virginica, Chloralum Hydratum, Cichorium, Cicuta Virosa, Cimex Lectularius, Cimicifuga Racemosa, Cina Maritima, Cinchona Off, Cinchoninum Sulphuricum, Cinnamomum, Clematis Erecta, Clematis Erecta, Cobaltum Metallicum, Coca-Erythroxylon Coca, Cocainum Hydrochloricum, Coccinella Septempunctata, Cocculus Indicus, Coccus Cacti, Cochlearia Armoracia, Codeinum, Coffea Cruda, Colchicum Autumnale, Collinsonia Canadensis, Colocynthinum, Colocynthis, Condurango, Conium Maculatum, Convallaria Majalis, Convolvulus Duartinus, Copaiva Officinalis, Corallium Rubrum, Cornus Circinata, Crataegus Oxyacantha, Crocus Sativus, Crotalus Cascavella, Crotalus Horridus, Croton Tiglium, Cundurango, Cupressus Australis, Cuprum Aceticum, Cuprum Metallicum, Curare, Cyclamen Europaeum, Cypripedium Pubescens, Daphne Indica, Digitalis Purpurea, Dioscorea Villosa, Dirca Palustris, Drosera Rotundifolia, Dulcamara, Echinacea Angustifolia, Eleeis Guineensis, Elaps Corallinus, Electricitas, Ephedra Vulgaris, Epiphegus Virginiana, Eserinum, Eucalyptus Globulus, Eugenia Jambos, Euonymus Atropurpurea, Euonymus Europea, Eupatorium Perfoliatum, Eupatorium Purpureum, Euphorbia Hypericifolia, Euphorbium Officinarum, Euphrasia Officinalis, Eupionum, Fagopyrum Esculentum, Fagus, Fel Tauri, Ferrum Bromatum, Ferrum Metallicum, Ferrum Muriaticum, Ferrum Phosphoricum, Ferrum Sulphuricum, Ferrum Tartaricum, Feverfew, Ficus Religiosa, Fluoricum Acidum, Formica Rufa, Franciscea Uniflora, Fraxinus Americana, Gallicum Acidum, Gaultheria Procumbens, Gelesemium Sempervirens, Gelsemium Sempervirens, Genista, Gentiana Lutea, Geranium Maculatum, Ginger, Ginseng, Ginseng Quinquefolium, Glonoine, Glonoinum, Glycerinum, Gnaphalium Polycephalum, Goldenseal, Gossypium Herbaceum, Graphites, Gratiola, Gratiola Officinalis, Grindelia Robusta, Guaco, Guaiacum, Guajacum Officinale, Guarana, Guarea Trichiloides, Gymnocladus Canadensis, Haematoxylon, Haematoxylon Campechianum, Hamamelis Virginiana, Hedeoma Pulegioides, Helleborus Niger, Heloderma, Helonias Dioica, Hepar Sulphur, Heracleum Sphondylium, Herbs Dandelion, Hippuricum Acidum, Homarus, Hydrastis Canadensis, Hydrocyanicum Acidum, Hydrophobinum, Hyoscyamus Niger, Hypericum, Hypericum Perforatum, Iberis Amara, Ichthyolum, Ictodes Foetida, Ignatia Amara, Indigo Tinctoria, Indium Metallicum, Indolum, Inula Helenium, Iodium, Iodoformum, Ipecacuanha, Iridium Metallicum, Iris Florentina, Iris Foetidissima, Iris Tenax, Iris Versicolor, Jacaranda Caroba, Joanesia Asoca, Juglans Cinerea, Juglans Regia, Juncus, Justicia Adhatoda, Kali Carbonicum, Kali Cyanatum, Kali Nitricum, Kali Oxalicum, Kali Phosphoricum, Kalium Bromatum, Kalium lodatum, Kalium Muriaticum, Kalium Silicicum, Kalium Sulphuricum, Kalmia Latifolia, Kreosotum, Lac Caninum, Lac Felinum, Lac Vaccinum Defloratum, Lachesis, Lachnanthes, Lactuca Virosa, Lamium, Lapathum, Latrodectus Mactans, Lecithinum, Ledum Palustre, Leptandra, Lilium Tigrinum, Limulus Cyclops, Lithium Carbonicum, Lobelia Cardinalis, Lobelia Dortmanna, Lobelia Inflata, Lobelia Purpurascens, Loleum Temulentum, Lonicera Xylosteum, Luna, Lupulus Humulus, Lycopersicum, Lycopodium Clavatum, Lycopus, Lyssinum, Macrotinum, Magnesia Muriatica, Magnesia Phosphorica, Magnesium Sulphuricum, Mancinella, Manganum, Medorrhinum, Melilotus Officinalis, Menispermum, Mentha Piperita, Mentha Pulegium, Mentholum, Menyanthes, Mercurialis Perennis, Mercurius Corrosivus, Mercurius Cyanatus, Mercurius Solubilis, Mezereum, Millefolium, Momordica Balsamica, Morphinum, Moschus, Muriaticum Acidum, Myrica Cerifera, Naja Tripudians, Natrium Arsenicosum, Natrium Nitricum, Natrium Phosphoricum, Natrium Salicylicum, Natrum Carbonicum, Natrum Hypochlorosum, Natrum Muriaticum, Natrum Sulphuricum, Niccolum Metallicum, Niccolum Sulphuricum, Nitricum Acidum, Nuphar Luteum, Nux Moschata, Nux Vomica, Nyctanthes Arbor Tristis, Oenanthe Crocata, Oleander, Oleum Animale Aethereum, Oleum Jecoris Aselli, Oniscus Asellus, Ononis, Onosmodium Virginianum, Opium, Oreodaphne, Osmium, Ostrya, Oxalicum Acidum, Oxytropis Lamberti, Paeonia, Palladium, Paraffinum, Paris, Paris Quadrifolia, Parthenium, Passiflora Incarnata, Penthorum Sedoides, Petroleum, Phaseolus, Phellandrium, Phenacetinum, Phosphoricum Acidum, Phosphorus, Physalis Alkekengi, Physostigma, Physostigma Venenosum, Phytolacca, Picricum Acidum, Pimpinella, Piper Methysticum, Piper Nigrum, Plantago Major, Platinum Metallicum, Plumbum Metallicum, Podophyllum, Polyporus, Populus Candicans, Pothos Foetida, Primula Veris, Prunus Padus, Prunus Spinosa, Psorinum, Ptelea, Pulex Irritans, Pulsatilla Pratensis, Pulsatilla, Pyrogenium, Radium Bromatum, Ranunculus Bulbosus, Ranunculus Glacialis, Ranunculus Sceleratus, Raphanus, Ratanhia Peruviana, Rhamnus Californica, Rheum, Rhodium Oxydatum Nitricum, Rhododendron Ferrugineum, Rhus Glabra, Rhus Toxicodendron, Rhus Venenata, Ricinus Communis, Robinia, Ruta Graveolens, Sabadilla, Sabal Serrulata, Sabina, Saccharum Lactis, Saccharum Officinale, Salicylicum Acidum, Salix Alba, Salol, Sambucus Nigra, Sanguinaria Canadensis, Sanguinarinum Nitricum, Sanicula, Santoninum, Saponaria Officinalis, Saponinum, Sarsaparilla, Scrophularia Nodosa, Scutellaria Laterifolia, Secale Cornutum, Selenium, Senecio Aureus, Senecio Jacoboea, Senega, Sepia Officinalis, Silica, Silicea Terra, Sinapis Alba, Sinapis Nigra, Skatolum, Sol, Solanum Nigrum, Solanum Tuberosum grotans, Spigelia Anthelmia, Spigelia Marilandica, Spongia Tosta, Stachys Betonica, Stannum, Staphysagria, Stellaria Media, Sticta Pulmonaria, Stillingia Sylvatica, Stramonium, Strontium Carbonicum, Strontium Nitricum, Strophanthus Hispidus, Strychninum, Succinum, Sulfonal, Sulphur, Sulphuricum Acidum, Sulphurosum Acidum, Sumbulus Moschatus, Symphytum Officinale, Syphilinum, Tabacum, Tanacetum Vulgare, Taraxacum Officinale, Tarentula Cubensis, Tarentula Hispanica, Taxus Baccata, Tellurium Metallicum, Terebinthiniae Oleum, Teucrium Marum Verum, Thea Chinensis, Theridion, Thlaspi Bursa Pastoris, Thuja Occidentalis, Thyroidinum, Tilia Europaea, Tongo, Torula Cerevisiae, Trifolium Pratense, Trillium Pendulum, Trinitrotoluenum, Triosteum Perfoliatum, Tuberculinum, Turnera Aphrodisiaca, Tussilago Petasites, Upas, Upas Tieut, Uranium Nitricum, Urtica Urens, Usnea Barbata, Ustilago, Ustilago Maydis, Vaccininum, Valeriana, Variolinum, Veratrum Album, Veratrum Nigrum, Veratrum Viride, Verbascum Thapsus, Viburnum Opulus, Vinca Minor, Viola Odorata, Viola Tricolor, Viscum Album, Wyethia, Xanthoxylum, Xerophyllum, X-Ray, Yohimbinum, Yucca Filamentosa, Zincum, Zincum Metallicum, Zincum Phosphoricum, Zincum Picricum, Zincum Sulphuricum, Zincum Valerianicum, Zingiber Officinale, Zizia, and any combination thereof.

The above ingredients, when making a formulation of the present invention, can be in any dilution (eg: 1X, 2X, 1C, 2C etc.). In an embodiment, the active agent or agents of the invention have been diluted so as to extract their active medicinal properties and to reach their therapeutically effective potency.elease their theraputically effective potency. In an embodiment, the dilution of the active ingredient is from about 1X to about 200C.

The formulations of the present invention are applied directly to the cephalic region (or head) of the host suffering from a headache. In an embodiment, the invention is applied to a region of the head selected from: the frontal region, the temporal region, the sphenoid region, the parietal region, the occipital region, the zygomatic region, and any combination thereof. In an embodiment, the invention is applied to the frontal and/or temporal regions (the forehead and/or temples). The active agents of the present invention are applied to a skin surface site of the cephalic region of the host. In an embodiment, the active agents of the invention are topically applied to an area proximal to the pain caused by the headache.

In an embodiment, the topical formulations are applied onto the head region of a host so as to be absorbed by the skin.

Treatment results in at least amelioration from one or more symptoms of the underlying condition, as described above, for a period of time that is variable depending on the severity of the symptom.

The active agents of the invention can be applied directly to the targeted site alone or in a mixture.

In an embodiment, the topical formulation of the present invention comprises at least one active agent listed above and a carrier. In an embodiment, the topical formulation of the present invention comprises at least one active agent listed above and at least one cooling agent. In an embodiment, the topical formulation of the present invention comprises at least one active agent listed above, at least one cooling agent and at least one carrier.

In an embodiment, any pharmaceutically acceptable carrier can be used in a mixture containing the active agents listed above. In an embodiment, the carrier is propylene glycol and/or ethyl alcohol. In an embodiment, the mixture includes approximately 80% (w/w) of propylene glycol and 10% (w/w) ethyl alcohol.

Any suitable cooling agents as defined herein can be used in combination with one or more of the active agents disclosed herein. In an embodiment, the cooling agents of the present invention include without limitation: menthol, ethyl alcohol, 2-Isopropyl-N,2,3-trimethylbutyramide, N-Ethyl-p-menthane-3-carboxamide, menthone glycerol ketal, (−)-Menthyl lactate, (−)-Menthoxypropane-1,2-diol, 3-(1-menthoxy)propane-1,2-diol, 3-(1-Menthoxy)-2-methylpropane-1,2-diol, Isopulegol, p-Menthane-3,8-diol, 2,3-dihydroxy-p-menthane, 3,3,5-trimethylcyclohexanone glycerol ketal, menthyl pyrrolidone carboxylate, (1R,3R,4S)-3-menthyl-3,6-dioxaheptanoate, (1R,2S,5R)-3-menthyl methoxyacetate, (1R,2S,5R)-3-menthyl 3,6,9-trioxadecanoate, (1R,2S,5R)-3-menthyl 3,6,9-trioxadecanoate, (1R,2S,5R)-3-menthyl (2-hydroxyethoxy)acetate, (1R,2S,5R)-menthyl 11-hydroxy-3,6,9-trioxaundecanoate, 1-[2-hydroxyphenyl]-4-[2-nitrophenyl-]-1,2,3,6-tetrahydropyrimidine-2-one, 5-methyl-4-(1-pyrrolidinyl)-3-[2H]-furanone, 4,5-dimethyl-3-(1-pyrrolidinyl)-2[5H]-furanone, 4-methyl-3-(1-pyrrolidinyl)-2[5H]-furanone, 4-methyl-3-(1-pyrrolidinyl)-2[5H]-furanone and combinations thereof.

The percentage of active agents of the topical formulations of the invention can be from about 0.001% (w/w) to about 99% (w/w).

The topical formulations of the present invention can be provided in the form of a cream, an ointment, a lotion, a balm, a salve, an emollient, a liniment, an unguent, a demulcent, a cerate, a foam, a liquid, a viscous liquid and a combination thereof.

In an embodiment, the present invention can be provided in a kit form. This kit includes a container, which includes one or more of the homeopathic ingredients listed above. The homeopathic ingredients may be provided in a mixture with a carrier or a cooling agent or a carrier and a cooling agent. In an embodiment, the container is a canister such as that used for a chap stick or a deodorant stick. In an embodiment, the active agent or agents are in the form of a balm. In an embodiment, this balm is in a stick form contained within a canister or tube or other suitable container for balms (such as that used for a chap stick or a deodorant stick) and applied to a head region such as the forehead for example.

The following is a non-limiting example of preparing a topical formulation in accordance with an embodiment of the invention.

Select a given active agent or agents from any of the homeopathic ingredients listed herein. Place this homeopathic ingredient or ingredients within a container. Add a carrier, such as propylene glycol into the container. Start moderate agitation and heat to about 75° C.(167° F.). Add distilled water and Tetrasodium EDTA and continue mixing until Tetrasodium EDTA is dissolved. Accelerate the agitation and add Sodium Stearate. Heat up to 80° C. (176° F.). Mix for 20 minutes or until the Sodium Stearate is completely dissolved Add Triethanolamine, Diazolidinyl Urea, Iodopropynyl Butylcarbamate, and menthol as a first cooling agent. Keep temperature between 75 to 80° C. (167-176° F.). Mix with medium agitation for about 20 minutes. Cool down to 60 to 65° C. (140-149° F.). In a premix tank dissolve menthone glycerol ketal, used here as a second cooling agent, in SD Alcohol 40B 190% proof. With batch tank at 60-65° C. (140-149° F.), add premix. Mix with medium agitation until product is homogeneous.

The invention will now be further described by way of non-limiting examples whose purpose is only to exemplify specific embodiments of the invention and not to limit the scope thereof.

### EXAMPLES

### Example 1

A stick containing the homeopathic active ingredients: Kali Bichromicum 6X H.P.U.S. (0.03%) and Bryonia Alba 12X H.P.U.S. (0.04%) was applied using a roll-on tube to the forehead of a patient (female, 32 years old) suffering from a tension headache. Patient complained of constant pressure on both sides of the head and throbbing in vertex, fullness, especially in forehead, also tenseness or stiffness around the neck. The product was applied and spreadout topically onto the forehead. The product was absorbed into the skin of the forehead leaving very little to no residue. Product was translucent and could not be seen by others. Within 15 minutes of application, patient stated intensity of headache was significantly reduced. Patient's normal medication consisted of tablets and other ingestibles. The topical homeopathic headache treatment provided faster and safer relief for the patient as compared to her normal headache medication.

### Example 2

A balm containing the homeopathic active ingredient: Iris Versicolor 6X H.P.U.S. (0.03%) was applied topically to a patient (female, 41 years old) suffering from a migraine headache. Patient complained of periodically recurring headache usually lasting 12 to 36 hours. Pain was pulsating and often, but not always, on one side of the head and would become worse with movement or physical exertion. Often patient would be sensitive to light and would become nauseous. Patient's symptoms were consistent with the diagnosis of migraine. The product was applied topically to her forehead on several occasions during a migraine attack. Product would leave no visible cosmetic trace on patient's forehead. Patient stated pain was alleviated partially but not completely after 11 minutes. Patient stated pain was completely alleviated in 19 minutes. This topical homeopathic headache treatment provided a more effective pain remedy for the patient as compared to ingestibles or cold compresses, which the patient regularly used.

### Example 3

A balm in a roll-on stick containing the homeopathic active ingredients: Sanguinaria Canadensis 12X H.P.U.S. (3.95%), Passiflora Incarnata 8C H.P.U.S. (1.86%) and Belladonna 1C H.P.U.S. (15.81%) was applied directly to the skin of the forehead and temples a patient (female, 46 years old) suffering from chronic headaches. Patient described her pain as dull and persistent, felt on both sides as well as on the back of her head. The product was applied topically in a uniform manner. Complete absorption of the balm occurred in approximately 2 minutes. Product was transparent and could not be seen by others. Within 11 minutes of application, patient attested to the significant reduction of pain intensity on both sides of the head. Within 14 minutes, patient stated pain was also significantly reduced on the back of her head. Within 22 minutes, patient stated her pain was now of a very low intensity on all parts of the head. The topical homeopathic headache treatment was effective at relieving the pain associated with patient's headache.

### Example 4

A topical ointment containing the homeopathic active ingredients: Ferrum phosphoricum 30X H.P.U.S. (5.0%), Cimicifuga Racemosa 30C H.P.U.S. (14.1%), Digitalis purpurea 8X H.P.U.S. (24.8%) and Spigelia anthelmia 10C H.P.U.S. (4.0%) was applied topically using the fingers to a patient (male, 29 years old) suffering from a migraine headache. Patient complained of moderate to severe throbbing pain often accompanied by a feeling of nausea, cold hands, sensitivity to light and sound and periodic vomiting. Patient was accustomed to taking internal pain relievers both by prescription and over-the-counter. Patient claimed these medications caused him to suffer from stomach ulcers with frequent usage. Patient stated he could feel intensity of pain beginning to decrease during fourth minute after application of topical headache treatment. Patient stated pain was alleviated completely within 13 minutes and that he was no longer sensitive to light and sound. Patient used same topical treatment on several occasions that migraines appeared and he claimed the same efficacy. Patient also stated that by not having to take internal pain relievers, he could avoid stomach ulcers entirely. The topical homeopathic headache treatment provided safer, more effective pain relief than his current internal medications.

It is to be understood that the invention is not limited in its application to the details of the examples described hereinabove. The invention is capable of other embodiments and of being practised in various ways. It is also to be understood that the phraseology or terminology used herein is for the purpose of description and not limitation. Hence, although the present invention has been described hereinabove by way of embodiments thereof, it can be modified, without departing from the spirit, scope and nature of the subject invention as defined in the appended claims.

1-24. (canceled)

25. A topical formulation, for treating, ameliorating or at least reducing pain intensity associated with a headache or a migraine in a host suffering therefrom, said formulation applicable to the cephalic region of said host, said formulation comprising: a therapeutically effective amount of an active agent, said active agent comprising Bryonia Alba, and a pharmaceulically acceptable carrier.

26. A topical formulation according to claim 25, wherein said active agent is so diluted as to release the therapeutically effective potency thereof.

27. A topical formulation according to claim 26, wherein said dilution is from about 1X to about 200C.

28. (canceled)

29. A topical formulation according to claim 25, wherein said pharmaceutically acceptable carrier comprises propylene glycol.

30. A topical formulation according to claim 25, wherein said formulation further comprises a cooling agent.

31-33. (canceled)

34. A topical formulation according to claim 25, wherein said active agent is present in said formulation from about 0.001% to about 99% w/w.

35-36. (canceled)

37. A topical formulation according to claim 25 in the form of a preparation.

38. (canceled)

39. A topical formulation according to claim 37, wherein said preparation comprises a balm in a stick form.

40-54. (canceled)

55. A topical formulation according to claim 25, wherein said pharmaceutically acceptable carrier comprises ethyl alcohol.

56. A topical formulation according to claim 25, wherein said active agent comprises a therapeutically effective amount of at least one additional homeopathic active ingredient in a mixture with Bryonia Alba.

57. A topical formulation according to claim 56, wherein said additional active ingredient comprises Kali Bichronium.

58. A topical formulation according to claim 56, wherein said additional active ingredient comprises Iris Versicolor.

59. A method according to claim 56, wherein said additional active ingredient comprises Hydrastis Canadensis.
